(19) United States
(12) Patent Application Publication
(10) Pub. No.: US 2026/0328712 A1
(43) Pub. Date: Oct. 22, 2026

(54) METHOD OF EVALUATING COMPOSITE REDUCING AGENT FOR RECYCLED MATERIAL

(71) Applicants: HYUNDAI MOTOR COMPANY, Seoul (KR); KIA CORPORATION, Seoul (KR); HM SOLUTION CO., LTD., Gyeongsan-si (KR)

(72) Inventors: Kwang Mo Seong, Hwaseong-si (KR); Jung Gyun Noh, Hwaseong-si (KR); In Soo Han, Hwaseong-si (KR); Eun Ju Noh, Hwaseong-si (KR); Tae Hwan Son, Yeongcheon-si (KR); Young Hoon Han, Gyeongsan-si (KR); Jin Won Park, Gyeongsan-si (KR)

(73) Assignees: HYUNDAI MOTOR COMPANY, Seoul (KR); KIA CORPORATION, Seoul (KR); HM SOLUTION CO., LTD., Gyeongsan-si (KR)

(21) Appl. No.: 19/371,792

(22) Filed: Oct. 28, 2025

(30) Foreign Application Priority Data
Apr. 18, 2025 (KR) 10-2025-0050951

(51) Int. Cl. G01N 23/223

(52) U.S. Cl. CPC G01N 23/223

(57) ABSTRACT

A method of evaluating a composite reducing agent for a recycled material is disclosed. The method includes obtaining a recycled material by applying and evaluating a reducing material corresponding to an odor generated from a target material and emitted volatile organic compounds, where an appropriate amount of the reducing material is used in the recycled material.

### CROSS-REFERENCE TO RELATED APPLICATION

This application claims, under 35 U.S.C. § 119 (a), the benefit of and priority to Korean Patent Application No. 10-2025-0050951, filed on Apr. 18, 2025, the entire contents of which are incorporated herein by reference.

### TECHNICAL FIELD

The present disclosure relates to a method of evaluating a composite reducing agent for a recycled material, particularly to a method of applying and evaluating a reducing material corresponding to odor generated from a target material and emitted volatile organic compounds.

### BACKGROUND

As part of the international response to climate change, various greenhouse gas reduction plans are being promoted with the goal of achieving carbon neutrality by 2050, and one thereof is the establishment of a circular economy (use of recycled materials/biomaterials). Accordingly, there is demand for expanded use of materials recycled from waste plastics, and in particular, the EU ELVR (circularity and end-of-life vehicle regulation) draft is promoting the expanded use of recycled raw materials in the automobile industry.

Recycling of waste plastics may be broadly divided into mechanical recycling and chemical recycling. Mechanical recycling is practical because the process is simple and economical, but there may be limitations to the scope of use due to some characteristic changes that may occur during the recycling process, and thus, thorough technological development is underway to solve such limitations. In particular, in order to expand the application to interior parts, it is important to ensure quality suitable for the usage environment, and for this purpose, it is necessary to control the level of odor and volatile organic compounds (VOCs).

Existing odor/VOCs reduction methods include the use of various process improvement methods such as washing, drying, vacuum venting, application of supercritical fluids, etc., and also the use of reducing agents such as adsorbents, etc. However, changes in process conditions or the addition of specific materials may affect the properties of recycled materials, necessitating a more precise and efficient approach.

### SUMMARY

The embodiments disclosed herein are intended to increase the usability of recycled materials, particularly to achieve improvements in odor and VOCs quality to a level suitable for application to interior parts.

An aspect of the present disclosure is to provide a method of evaluating a composite reducing agent for a recycled material capable of effectively improving quality by analyzing causal substances of odor generated from a target material to be recycled and emitted VOCs and providing a composite reducing agent that targets the causal substance and VOCs.

Another aspect of the present disclosure is to provide a verification method using a tracer material to enable quantitative/qualitative evaluation of the use of the composite reducing agent, thereby providing odor/VOCs reduction technology that takes into account the balance between productivity and quality.

The application of the disclosed embodiments should enable the widespread adoption of interior parts made of recycled materials, strengthening the establishment of a circular economy and contributing to the realization of carbon neutrality.

The aspects of the present disclosure are not limited to the foregoing. The aspects of the present disclosure should be clearly understood through the following description and should be realized by the means described in the claims and combinations thereof.

An aspect of the present disclosure provides a method of evaluating a composite reducing agent for a recycled material, including determining a reducing material to be applied to a target material, manufacturing a composite reducing agent by mixing the reducing material and a tracer material, determining an amount of the composite reducing agent to be added to the target material, manufacturing a recycled material by mixing the target material and the composite reducing agent, and analyzing the composite reducing agent in the recycled material.

In an embodiment of the present disclosure, the target material may include a waste polymer material.

In an embodiment of the present disclosure, determining the reducing material may include determining a causal substance of odor generated from the target material and an emitted volatile organic compounds (VOCs) and determining a reducing material corresponding to the determined causal substance and volatile organic compounds.

In an embodiment of the present disclosure, determining the causal substance of odor and the emitted volatile organic compounds may include determining the causal substance of odor and the volatile organic compounds as at least one of aldehydes, amines, ketones, aromatic hydrocarbons, sulfur compounds, or fatty acids based on a chemical composition of the causal substance of odor and the volatile organic compounds.

In an embodiment of the present disclosure, determining the reducing material may include selecting one type of reducing material or determining a mixing ratio of two or more types of reducing materials.

In an embodiment of the present disclosure, determining the mixing ratio of the reducing materials may include mixing the two or more types of reducing materials, mixing the target material with the reducing materials, and determining the mixing ratio based on results of an odor evaluation for the mixed target material and the reducing materials.

In an embodiment of the present disclosure, the reducing material may include at least one of a physical adsorbent or a chemical adsorbent.

In an embodiment of the present disclosure, the reducing material may include at least one of Zeolite 3,4A type, Zeolite X(Y) type, activated carbon, citric acid, piperazine, or peroxyacetic acid.

In an embodiment of the present disclosure, the Zeolite 3,4A type and piperazine may correspond to an aldehyde-based causal substance and volatile organic compounds, the Zeolite X(Y) type may correspond to an amine- and ketone-based causal substance and volatile organic compounds, the activated carbon may correspond to an aromatic hydrocarbon-based causal substance and volatile organic compounds, the citric acid may correspond to an amine-based causal substance and volatile organic compounds, and the peroxyacetic acid may correspond to a ketone-based causal substance and volatile organic compounds.

In an embodiment of the present disclosure, the tracer material may be selected from among metals having a molecular weight greater than carbon (C) and oxides of the metals.

In an embodiment of the present disclosure, the tracer material may include at least one of ZrO₂ or MnO₂.

In an embodiment of the present disclosure, determining the amount of the composite reducing agent may include mixing the target material and the composite reducing agent and determining the amount of the composite reducing agent based on results of an odor and volatile organic compounds evaluation for the mixed target material and the composite reducing agent.

In an embodiment of the present disclosure, manufacturing the recycled material may include adding the composite reducing agent to at least one of an extrusion process or a compounding process for the target material.

In an embodiment of the present disclosure, analyzing the composite reducing agent in the recycled material may include pretreating the recycled material.

In an embodiment of the present disclosure, pretreating the recycled material may include processing the recycled material into a specimen by mixing ash remaining after heat treatment of the recycled material with a binder.

In an embodiment of the present disclosure, analyzing the composite reducing agent in the recycled material may include determining whether the composite reducing agent is added through qualitative analysis of the recycled material.

In an embodiment of the present disclosure, analyzing the composite reducing agent in the recycled material may include determining the amount of the composite reducing agent through quantitative analysis of the recycled material.

In an embodiment of the present disclosure, analyzing the composite reducing agent in the recycled material may be performed using wavelength dispersive X-ray fluorescence (WD-XRF) or energy dispersive X-ray fluorescence (ED-XRF).

In an embodiment of the present disclosure, analyzing the composite reducing agent in the recycled material may include analyzing the tracer material in the recycled material and analyzing the reducing material based on analysis results of the tracer material.

In an embodiment of the present disclosure, analyzing the composite reducing agent in the recycled material may include performing qualitative analysis of the recycled material using energy dispersive X-ray fluorescence, processing the recycled material into a specimen by pretreatment, and performing quantitative analysis of the specimen using wavelength dispersive X-ray fluorescence.

### BRIEF DESCRIPTION OF THE DRAWING

FIG. 1 shows various embodiments of an evaluation process according to an embodiment of the present disclosure.

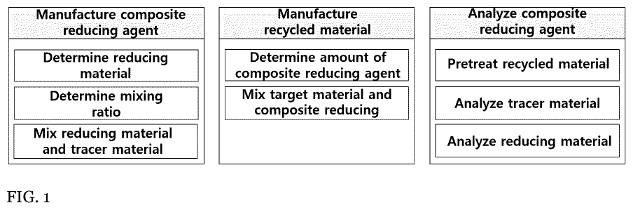

### DETAILED DESCRIPTION

The above and other aspects, features and advantages of the present disclosure should be more clearly understood from the following embodiments taken in conjunction with the accompanying drawing. However, the present disclosure is not limited to the embodiments disclosed herein, and may be modified into different forms. These embodiments are provided to thoroughly explain the disclosure and to sufficiently transfer the spirit of the present disclosure to those having ordinary skill in the art.

Throughout the drawing, the same reference numerals may refer to the same or like elements. For the sake of clarity of the present disclosure, the dimensions of structures may be depicted as being larger than the actual sizes thereof. It should be understood that, although terms such as “first”, “second”, etc. may be used herein to describe various elements, these elements are not to be limited by these terms. These terms are only used to distinguish one element from another element. For instance, a “first” element discussed below could be termed a “second” element without departing from the scope of the present disclosure. Similarly, the “second” element could also be termed a “first” element. As used herein, the singular forms are intended to include the plural forms as well, unless the context clearly indicates otherwise.

It should be further understood that the terms “comprise”, “include”, “have”, etc., when used in this specification, specify the presence of stated features, integers, steps, operations, elements, components, or combinations thereof, but do not preclude the presence or addition of one or more other features, integers, steps, operations, elements, components, or combinations thereof. Also, it should be understood that when an element such as a layer, film, area, or sheet is referred to as being “on” another element, it may be directly on the other element, or intervening elements may be present therebetween. Similarly, when an element such as a layer, film, area, or sheet is referred to as being “under” another element, it may be directly under the other element, or intervening elements may be present therebetween.

Unless otherwise specified, all numbers, values, and/or representations that express the amounts of components, reaction conditions, polymer compositions, and mixtures used herein are to be taken as approximations including various uncertainties affecting measurement that inherently occur in obtaining these values, among others, and thus should be understood to be modified by the term “about” in all cases. Furthermore, when a numerical range is disclosed in this specification, the range is continuous, and includes all values from the minimum value of said range to the maximum value thereof, unless otherwise indicated. Moreover, when such a range pertains to integer values, all integers including the minimum value to the maximum value are included, unless otherwise indicated.

In the present specification, when a range is described for a variable, it should be understood that the variable includes all values including the end points described within the stated range. For example, the range of “5 to 10” should be understood to include any subranges, such as 6 to 10, 7 to 10, 6 to 9, 7 to 9, and the like, as well as individual values of 5, 6, 7, 8, 9 and 10, and will also be understood to include any value between valid integers within the stated range, such as 5.5, 6.5, 7.5, 5.5 to 8.5, 6.5 to 9, and the like. Also, for example, the range of “10% to 30%” should be understood to include subranges, such as 10% to 15%, 12% to 18%, 20% to 30%, etc., as well as all integers including values of 10%, 11%, 12%, 13% and the like up to 30%, and will also be understood to include any value between valid integers within the stated range, such as 10.5%, 15.5%, 25.5%, and the like.

Furthermore, unless specifically stated otherwise, the term “about” as used herein may be understood within a range of error that is typical in the art (e.g., within 2 standard deviations of the mean). “About” may be understood as within 10%, 9%, 8%, 7%, 6%, 5%, 4%, 3%, 2%, 1%, 0.5%, 0.1%, 0.05%, or 0.01% of the stated value.

In addition, “vehicle” or “automobile” or other similar terms used herein are understood to include general automobiles such as sport utility vehicles (SUVs), buses, trucks, various commercial vehicles, etc., and transportation means such as trains, ships, and aircraft, and also include hybrid vehicles, electric vehicles, plug-in hybrid electric vehicles, hydrogen-powered vehicles, and other alternative fuel vehicles (e.g., fuel derived from resources other than petroleum). “Hybrid vehicle” as used herein means a vehicle that has two or more power sources (e.g., a gasoline-powered vehicle and an electric-powered vehicle).

When a certain computational ability is required to perform any method or program herein, it may be understood that it is driven by a hardware device that includes a memory and a processor and is specifically programmed to execute the method or program described herein. The memory is configured to store a module, and the processor is specifically configured to execute the module to perform one or more processes as further described below.

The method or program may be implemented on or as a non-transitory computer readable medium on a computer readable medium including executable program instructions executed by a processor, controller, etc. Examples of the computer readable medium include, but are not limited to, ROM, RAM, compact disc (CD)-ROM, magnetic tape, floppy disks, flash drives, smart cards, and optical data storage devices. The computer readable medium may also be distributed to networked computer systems for storage and execution in a distributed manner, such as a telematics server or a CAN (controller area network).

A method of evaluating a composite reducing agent for a recycled material according to an aspect of the present disclosure may include determining a reducing material to be applied to a target material, manufacturing a composite reducing agent by mixing the reducing material and a tracer material, determining the amount of the composite reducing agent to be added to the target material, manufacturing a recycled material by mixing the target material and the composite reducing agent, and analyzing the composite reducing agent in the recycled material.

In an embodiment of the present disclosure, the target material may include a waste polymer material. The waste polymer material includes a polymer compound and may refer to a scrap that has already been manufactured into a product and has reached end of life, has been manufactured but has been judged to be defective, or is generated during the process of manufacturing a product. According to the disclosed embodiment, as the waste polymer material may be used as a target material, resource recycling and environmental sustainability may be promoted.

In an embodiment of the present disclosure, the target material may be the waste polymer material subjected to at least one process of recovery, selection, crushing, washing, or extrusion, as needed.

In an embodiment of the present disclosure, determining the reducing material may include determining a causal substance of odor (i.e., a substance that causes an odor) generated from the target material and emitted volatile organic compounds (VOCs) and determining a reducing material corresponding to the determined causal substance and volatile organic compounds.

Determining the causal substance of odor and the emitted volatile organic compounds may include analyzing the chemical composition of the causal substance and the volatile organic compounds using a known analysis method for the target material, such as gas chromatography/mass spectrometry (GC/MS), gas chromatography/flame ionization detector (GC/FID), high-performance liquid chromatography (HPLC), ultraviolet/visible spectroscopy (UV/VIS), etc.

In an embodiment of the present disclosure, determining the causal substance of odor and the emitted volatile organic compounds may include determining the causal substance and the volatile organic compounds as at least one of aldehydes, amines, ketones, aromatic hydrocarbons, sulfur compounds, or fatty acids based on the chemical composition of the causal substance and the volatile organic compounds.

In addition, based on the result of analyzing the chemical composition of the volatile organic compounds indicating that the content of a certain type is equal to or less than a predetermined reference content, for example, the emission regulation standard of volatile organic compounds presented by organizations such as the Ministry of Land, Infrastructure and Transport, the Ministry of Environment, etc., evaluation for the volatile organic compounds determined to be equal to or less than the reference content may be omitted in the following process.

According to the disclosed embodiment, a reducing material corresponding to the determined causal substance and volatile organic compounds may be determined. The term “reducing material corresponding to the determined causal substance and volatile organic compounds” may be understood to mean a physical adsorbent, a chemical adsorbent, or a combination thereof capable of easily removing or reducing the amount of the determined causal substance and volatile organic compounds in consideration of the molecular size of the causal substance and volatile organic compounds. In addition to the types presented below, various other materials may be used.

In an embodiment of the present disclosure, the reducing material may include at least one of Zeolite 3,4A type, Zeolite X(Y) type, activated carbon, citric acid, piperazine, or peroxyacetic acid.

In an embodiment of the present disclosure, the Zeolite 3,4A type and piperazine may correspond to aldehyde-based causal substances and volatile organic compounds. The Zeolite X(Y) type may correspond to amine- and ketone-based causal substances and volatile organic compounds. The activated carbon may correspond to aromatic hydrocarbon-based causal substances and volatile organic compounds. The citric acid may correspond to amine-based causal substances and volatile organic compounds. The peroxyacetic acid may correspond to ketone-based causal substances and volatile organic compounds. In one example, the activated carbon has a BET surface area of 1,000 m²/g or more.

In an embodiment of the present disclosure, depending on the causal substance of odor and volatile organic compounds to be reduced, one type of reducing material may be used alone, or two or more types of reducing materials may be used in combination. As such, the two or more types of reducing materials may be used by impregnating any reducing material as a support with another reducing material, or by simply mixing reducing materials, depending on the form thereof.

In an embodiment of the present disclosure, determining the reducing material may include selecting one type of reducing material or, based on two or more types of reducing materials being selected, determining a mixing ratio of the two or more types of reducing materials. Determining the mixing ratio of the reducing materials may include mixing two or more types of reducing materials in any ratio, mixing the target material and the reducing materials, and determining the mixing ratio based on results of an odor evaluation for the mixed target material and the reducing materials.

Based on results of the odor evaluation for the mixture, the mixing ratio with the greatest odor reduction may be adopted compared to results of an odor evaluation for a target material without a reducing material added.

Any method of mixing the target material and the reducing material may be used so long as it is possible to manufacture a mixture in a form in which evaluation of odor is easy, and for example, a method such as melt mixing using an extruder or dry mixing using a milling machine may be applied. In addition, odor evaluation for the mixture may be performed using a method such as the known analysis method or sensory evaluation.

In the disclosed embodiment, the composite reducing agent may be manufactured by mixing the determined reducing material and the tracer material. The tracer material may be selected from among metals having a molecular weight greater than carbon (C) and oxides of the metals. In one example, the tracer material includes at least one of ZrO₂ or MnO₂.

According to the disclosed embodiment, the composite reducing agent may be manufactured by mixing the reducing material and the tracer material, and by performing quantitative or qualitative analysis of the recycled material subjected to a pretreatment process such as heat treatment, the amount of the reducing material added may be determined with high reproducibility based on the amount of the tracer material.

In an embodiment of the present disclosure, the amount of the tracer material in the composite reducing agent is not particularly limited, and may be, for example, 0.5 wt % to 3 wt % based on the total weight of the composite reducing agent.

According to the disclosed embodiment, the amount of the composite reducing agent that is added to the target material may be determined. In an embodiment of the present disclosure, determining the amount of the composite reducing agent may include mixing the target material and the composite reducing agent and determining the amount of the composite reducing agent based on results of an odor and volatile organic compounds evaluation for the mixed target material and the composite reducing agent.

By comparing the results of evaluation for the mixture including the target material and the composite reducing agent with the results of odor and volatile organic compounds evaluation for the target material without the reducing material added, it is possible to adopt the amount of the composite reducing agent in which the content and grade of the causal substance and the content of the volatile organic compounds are reduced in a balanced manner.

Any method of mixing the target material and the composite reducing agent may be used so long as it is possible to manufacture a mixture in a form in which evaluation of odor is easy, and for example, a method such as melt mixing using an extruder or dry mixing using a milling machine may be applied. In addition, odor evaluation for the mixture may be performed using a method such as the known analysis method or sensory evaluation.

In order to verify the effect depending on the determined amount of the composite reducing agent from more diverse aspects, the target material and a virgin polymer that has not been recycled are mixed, the resulting mixture is further mixed with the composite reducing agent to afford a mixture, and then the odor and volatile organic compound evaluation for the mixture may be performed.

According to the disclosed embodiment, a recycled material may be manufactured by mixing the target material and the composite reducing agent in the determined amount. In an embodiment of the present disclosure, manufacturing the recycled material may include adding the composite reducing agent to at least one of an extrusion process or a compounding process for the target material.

According to the disclosed embodiment, the composite reducing agent in the recycled material may be analyzed. In an embodiment of the present disclosure, analyzing the composite reducing agent in the recycled material may include pretreating the recycled material. The pretreatment may include processing the recycled material into a specimen by mixing ash remaining after heat treatment of the recycled material with a binder.

In an embodiment of the present disclosure, analyzing the composite reducing agent in the recycled material may include performing qualitative analysis or quantitative analysis of the recycled material. Determining whether the composite reducing agent is added through qualitative analysis of the recycled material may be included. Determining the amount of the composite reducing agent through quantitative analysis of the recycled material may be included.

In an embodiment of the present disclosure, analyzing the composite reducing agent in the recycled material may include analyzing the tracer material in the recycled material and analyzing the reducing material based on the analysis results of the tracer material.

In an embodiment of the present disclosure, analyzing the composite reducing agent in the recycled material may be performed using wavelength dispersive X-ray fluorescence (WD-XRF) or energy dispersive X-ray fluorescence (ED-XRF).

In an embodiment of the present disclosure, analyzing the composite reducing agent in the recycled material may include performing qualitative analysis of the recycled material using energy dispersive X-ray fluorescence, processing the recycled material into a specimen by pretreatment, and performing quantitative analysis of the specimen using wavelength dispersive X-ray fluorescence.

A better understanding of the present disclosure may be obtained through the following examples and comparative examples. However, these examples are not to be construed as limiting the technical spirit of the present disclosure.

### Example 1—Recycled Propylene from Waste Bumper

### Manufacture of Composite Reducing Agent

Waste bumpers containing polypropylene were crushed and washed to prepare a target material to be recycled (hereinafter referred to as “recycled propylene”).

In order to determine the causal substance of odor generated from the target material and emitted volatile organic compounds, quantitative analysis of eight types of VOCs (benzene, toluene, ethylbenzene, xylene, styrene, formaldehyde, acetaldehyde, and acrolein) and odor sensory evaluation for the recycled polypropylene were performed, and the results are shown in Table 1 below.

The quantitative analysis was performed using gas chromatography/mass spectrometry (GC/MS) and high-performance liquid chromatography (HPLC), and whether the analysis results satisfied the VOC emission regulation standards of the Ministry of Land, Infrastructure and Transport was determined. In addition, the odor sensory evaluation was conducted according to VDA 270, with five evaluators evaluating the odor intensity on a six-level scale from grade 1 (odorless) to grade 6 (unbearable odor) and then calculating the average value excluding the minimum and maximum values.

In order to specifically identify the causal substance of odor, additional quantitative analysis was performed using GC/MS and HPLC, and the results were classified based on aldehydes, amines, ketones, aromatic hydrocarbons, or others. The values for each classification were converted into percentages and are shown in Table 1 below.

TABLE 1

| Classification | | Analysis results |
|---|---|---|
| VOCs (8 types) | Xylene | 3868 μg/m³ |
| | The remaining | Satisfying regulation |
| | 7 types | standards |
| Odor | Grade (type) | 3.5 (burnt odor) |
| Odor contribution rate | Ketones | 51% |
| | Aldehydes | 9% |
| | Aromatics | 16% |
| | Others | 24% |

Referring to Table 1, when recycled propylene applied to waste bumpers was used as the target material, the causal substances of odor were confirmed to be ketones, aldehydes, and aromatic hydrocarbons.

The types of reducing materials corresponding to individual causal substances were determined. A Zeolite X(Y) type support impregnated with peroxyacetic acid was used as a reducing material corresponding to a ketone-based causal substance, a Zeolite 3,4A type support impregnated with piperazine was used as a reducing material corresponding to an aldehyde-based causal substance, and activated carbon having a BET surface area of 1,000 m²/g or more was used as a reducing material corresponding to an aromatic hydrocarbon-based causal substance.

The prepared ketone-corresponding reducing material, aldehyde-corresponding reducing material, and aromatic hydrocarbon-corresponding reducing material were mixed in a ratio as shown in Table 2 below and then mixed with the recycled polypropylene. As such, the amount of the reducing material was 2 wt % based on the total weight of the mixture. Thereafter, odor sensory evaluation for the mixture was performed using the same method as above.

TABLE 2

| | | | | | | | | | |
|---|---|---|---|---|---|---|---|---|---|
| Mix- | Ketones | Not | 80% | 60% | 50% | 40% | 40% | 40% | 30% |
| ing | Alde- | added | 10% | 20% | 10% | 40% | 20% | 10% | 20% |
| ratio | hydes | (Ref.) | | | | | | | |
| | Aro- | | 10% | 20% | 40% | 20% | 40% | 50% | 50% |
| | matics | | | | | | | | |

| | | | | | | | | |
|---|---|---|---|---|---|---|---|---|
| Odor (grade) | 4.0 | 3.6 | 3.4 | 3.4 | 3.3 | 3.2 | 3.3 | 3.5 |

Referring to Table 2, the mixing ratio of the reducing materials that showed the greatest odor reduction effect was determined to be 40% of the ketone-corresponding reducing material, 20% of the aldehyde-corresponding reducing material, and 40% of the aromatic hydrocarbon-corresponding reducing material.

The reducing materials having the mixing ratio determined above were prepared and then mixed with a tracer material including ZrO₂ and MnO₂, thereby manufacturing a composite reducing agent. The tracer material was added in an amount of 1 wt % based on 100 wt % of the composite reducing agent.

### Manufacture of Recycled Material

A typical extrusion molding machine equipped with a main hopper or a side hopper was prepared to manufacture a recycled material, and the recycled polypropylene and the composite reducing agent were fed as raw materials. Thereafter, a recycled material specimen was manufactured through extrusion and injection processes. In this example, the amount of the composite reducing agent added to the recycled polypropylene was set to 1 wt %, 2 wt %, 3 wt %, and 4 wt % based on 100 wt % of the recycled polypropylene.

Quantitative analysis of eight types of VOCs and odor sensory evaluation were performed on the manufactured recycled material specimen using the same method as above, and the results are shown in Table 3 below.

TABLE 3

| | | Not | 1.0 | 2.0 | 3.0 | 4.0 |
|---|---|---|---|---|---|---|
| Classification | | added | wt % | wt % | wt % | wt % |
| VOCs | Benzene | 26 | 24 | 21 | 23 | 22 |
| | Toluene | 43 | 51 | 39 | 36 | 30 |
| | Ethylbenzene | 133 | 79 | 68 | 59 | 40 |
| | Xylene | 1241 | 813 | 631 | 537 | 371 |
| | Styrene | 128 | 43 | 37 | 14 | 12 |
| | Formaldehyde | 41 | 26 | 24 | 23 | 21 |
| | Acetaldehyde | 282 | 251 | 171 | 170 | 152 |
| | Acrolein | 8 | 1 | Not | Not | Not |
| | | | | detected | detected | detected |
| Odor | Grade (type) | 4.0 | 3.5 | 3.5 | 3.0 | 3.0 |

Referring to Table 3, when 1 wt % of the composite reducing agent was added, xylene was reduced to be lower than the regulation standard (870 μg/m³), and odor was reduced to grade 3.5 but was still at a level where odor could be easily detected. When 3 wt % of the mixed reducing agent was added, all eight types of VOCs satisfied the regulation standards, and the odor was reduced to grade 3.0, a weakly detectable level.

In addition, in order to verify the effect of the composite reducing agent from more diverse aspects, as raw materials fed into an extrusion molding machine, 40 wt % of recycled polypropylene, 32 wt % of virgin polypropylene, 10 wt % of polyolefin elastomer (POE), and 18 wt % of talc were added and the composite reducing agent was added in amounts of 1.0 wt %, 1.5 wt %, and 2.0 wt % based on a total of 100 wt % thereof.

Quantitative analysis of eight types of VOCs and odor sensory evaluation were performed on the manufactured recycled material specimen using the same method as above, and the results are shown in Table 4 below.

TABLE 4

| Classification | | Not added | 1.0 wt % | 1.5 wt % | 2.0 wt % |
|---|---|---|---|---|---|
| VOCs | Benzene | 5 | 7 | 6 | 5 |
| | Toluene | 20 | 22 | 29 | 24 |
| | Ethylbenzene | 10 | 12 | 7 | 10 |
| | Xylene | 52 | 34 | 38 | 37 |
| | Styrene | 17 | 18 | 8 | 7 |
| | Formaldehyde | 742 | 163 | 66 | 46 |
| | Acetaldehyde | 164 | 55 | 32 | 27 |
| | Acrolein | 13 | 9 | 3 | 1 |
| Odor | Grade (type) | 3.5 | 3.5 | 3.0 | 3.0 |

Referring to Table 4, when 1.5 wt % of the composite reducing agent was added, all eight types of VOCs satisfied the regulation standards, and the odor was reduced to grade 3.0, a weakly detectable level.

### Analysis of Composite Reducing Agent—WD-XRF

A recycled material was manufactured according to the above procedure, and two test pieces were manufactured: a standard test piece for creation of a calibration curve and an evaluation test piece.

As such, the standard test piece was made of, as raw materials fed into an extrusion molding machine, 40 wt % of recycled polypropylene, 32 wt % of virgin polypropylene, 10 wt % of polyolefin elastomer (POE), 18 wt % of talc, and the composite reducing agent added in amounts of 1.0 wt %, 2.0 wt %, and 3.0 wt % based on a total of 100 wt % thereof, and the evaluation test piece was made of 40 wt % of recycled polypropylene, 32 wt % of virgin polypropylene, 10 wt % of polyolefin elastomer (POE), 18 wt % of talc, and the composite reducing agent added in amounts of 1.0 wt %, 1.5 wt %, and 2.0 wt % based on a total of 100 wt % thereof.

Each test piece thus manufactured was placed in a furnace and heat-treated at 600° C. for 2 hours. The ash remaining after heat treatment was recovered and mixed with a cellulose binder in a weight ratio of 4:1. The mixture was processed into a disc by pressing the same at about 193 bar for 90 seconds using a press.

A calibration curve was created through wavelength dispersive X-ray fluorescence analysis of the standard test piece processed into a disc, and the results are shown in Table 5 below.

TABLE 5

| | | Not | 1.0 | 2.0 | 3.0 |
|---|---|---|---|---|---|
| Classification | | added | wt % | wt % | wt % |
| Zr calibration | Theoretical value (ppm) | — | 181 | 353 | 534 |
| curve | Analyzed value (ppm) | 5.5 | 218.4 | 441.5 | 607.8 |
| | Coefficient of | 0.9976 | | | |
| | determination (R²) | | | | |
| Mn calibration | Theoretical value (ppm) | — | 234 | 456 | 691 |
| curve | Analyzed value (ppm) | 6.8 | 315.1 | 578.4 | 766.3 |
| | Coefficient of | 0.9912 | | | |
| | determination (R²) | | | | |

Referring to Table 5, based on the results of the calibration curve for each tracer material, both zirconium (Zr) and manganese (Mn) were found to have a very high coefficient of determination (Re) of 0.99 or more and to thus be suitable for use in the calibration curve.

In addition, the amounts of Zr and Mn were measured by quantitative analysis of the evaluation test piece processed into a disc, and reproducibility was calculated by comparing the same with predicted amounts of Zr and Mn using the created calibration curve. The results are shown in Table 6 below.

TABLE 6

| | Tracer | | | | | | | |
|---|---|---|---|---|---|---|---|---|
| | material | | | | | | | |
| Amount | theoretical | | WD-XRF quantitative analysis results | | | | | |
| of | value | | | Reducing | | | Reducing | |
| reducing | Zr | Mn | Zr | agent | | Mn | agent | |
| agent | (ppm) | (ppm) | (ppm) | amount | Reproducibility | (ppm) | amount | Reproducibility |
| Not | — | — | 21.0 | 0.13% | — | 33.3 | 0.16% | — |
| added | | | | | | | | |
| 1.0 wt % | 158 | 205 | 174.3 | 0.97% | 97% | 226.0 | 0.94% | 94% |
| 1.5 wt % | 236 | 306 | 254.0 | 1.48% | 99% | 335.1 | 1.48% | 99% |
| 2.0 wt % | 311 | 402 | 319.8 | 1.92% | 96% | 425.7 | 1.95% | 98% |

Referring to Table 6, reproducibility was obtained in a very high level of 96-99% in the presence of Zr as a tracer material and 94-99% in the presence of Mn as a tracer material. Based on these results, it is expected that the amount of the reducing agent added to the recycled material may be confirmed by sequentially performing manufacture of the composite reducing agent with the tracer material added, application thereto to the recycled material, pretreatment of the recycled material, and quantitative analysis of the tracer material.

### Analysis of Composite Reducing Agent—ED-XRF

A standard test piece and an evaluation test piece were manufactured as in the quantitative analysis of the composite reducing agent. The standard test piece was processed into a disc by pretreatment using the same method as above, and the evaluation test piece was not pretreated.

A calibration curve was created through portable energy dispersive X-ray fluorescence (ED-XRF) of the standard test piece processed into a disc. In addition, the amounts of Zr and Mn were measured by quantitative analysis using ED-XRF in a non-destructive state of the evaluation test piece not subjected to any pretreatment, and reproducibility was calculated by comparing the same with predicted amounts of Zr and Mn using the created calibration curve. The results are shown in Table 7 below.

TABLE 7

| | Tracer | | | | | | | |
|---|---|---|---|---|---|---|---|---|
| | material | | | | | | | |
| Amount | theoretical | | ED-XRF quantitative analysis results | | | | | |
| of | value | | | Reducing | | | Reducing | |
| reducing | Zr | Mn | Zr | agent | | Mn | agent | |
| agent | (ppm) | (ppm) | (ppm) | amount | Reproducibility | (ppm) | amount | Reproducibility |
| Not | — | — | 8 | 0.08% | — | 0 | — | — |
| added | | | | | | | | |
| 1.0 wt % | 158 | 205 | 23 | 0.09% | 9% | 11 | 0.05% | 5% |
| 1.5 wt % | 236 | 306 | 42 | 0.22% | 14% | 33 | 0.16% | 11% |
| 2.0 wt % | 311 | 402 | 54 | 0.30% | 15% | 55 | 0.27% | 14% |

Referring to Table 7, reproducibility obtained by portable ED-XRF was determined to be 9-15% in the presence of Zr and 5-14% in the presence of Mn, which were lower than the results of WD-XRF. Based on these results, it is judged that ED-XRF is more appropriate for qualitative analysis rather than quantitative analysis of the tracer material. In terms of practical use, it is expected that quality control is possible by determining whether the reducing agent was used through portable ED-XRF in the field and finally confirming the added amounts of tracer material and reducing agent through WD-XRF quantitative analysis of recycled materials or recycled material parts where the use of the reducing agent is suspected.

### Example 2—Recycled Polyamide 6 from Waste Fishing Net

### Manufacture of Composite Reducing Agent

Waste fishing nets containing polyamide 6 were crushed and washed to prepare a target material to be recycled (hereinafter referred to as “recycled PA6”).

In order to determine the causal substance of odor generated from the target material and emitted volatile organic compounds, quantitative analysis of eight types of VOCs and odor sensory evaluation were performed on the recycled PA6 using the same method as in Example 1, and the results are shown in Table 8 below.

In addition, to specifically identify the causal substance of odor, TMA (trimethyl amine), which may cause fishy and salty odor, was quantitatively analyzed using gas chromatography/flame ionization detector (GC/FID), and ammonia was quantitatively analyzed using ultraviolet/visible spectroscopy (UV/VIS), and the results are shown in Table 8 below.

TABLE 8

| | |
|---|---|
| Classification | Analysis results |

| | | |
|---|---|---|
| VOCs (8 types) | All | Satisfying regulation standards |
| Odor | Grade (type) | 3.5 (fishy and salty odor) |
| | TMA | 33 μg/m³ |
| | Ammonia | 418 μg/m³ |

Referring to Table 8, all eight types of VOCs satisfied the regulation standards of the Ministry of Land, Infrastructure and Transport. The odor was determined to be grade 3.5 as a level where fishy/salty odor could be easily detected based on the sensory evaluation results, and TMA (trimethyl amine) and ammonia were detected in high concentrations.

Thus, the causal substance of odor was confirmed to be amines when recycled PA6 was used as the target material.

The type of reducing material corresponding to the causal substance was determined. The amine-corresponding reducing material was used alone, and specifically, a Zeolite X(Y) type support impregnated with citric acid was used.

Since the amine-corresponding reducing material was used alone as the reducing material, determining the mixing ratio was omitted, unlike Example 1.

The reducing material determined above was manufactured and mixed with a tracer material including ZrO₂ and MnO₂, thereby manufacturing a composite reducing agent. The tracer material was added in an amount of 1 wt % based on 100 wt % of the composite reducing agent.

### Manufacture of Recycled Material

To manufacture a recycled material, a typical extrusion molding machine equipped with a main hopper or a side hopper was prepared, and the recycled PA6 and the composite reducing agent were fed as raw materials. Then, a recycled material specimen was manufactured through extrusion and injection processes. In this example, the amount of the composite reducing agent added to the recycled PA6 was set to 0.5 wt %, 1.0 wt %, and 2.0 wt % based on 100 wt % of the recycled PA6.

Odor sensory evaluation and quantitative analysis were performed on the manufactured recycled material specimen using the same method as above, and the results are shown in Table 9 below. Since all eight types of VOCs satisfied the regulation standards in Table 8, no additional testing was performed.

TABLE 9

| Classification | | 0.5 wt % | 1.0 wt % | 2.0 wt % |
|---|---|---|---|---|
| VOCs | 8 types | — | | |
| Odor | Grade (type) | 3.5 | 3.0 | 3.0 |
| | | (fishy and salty odor) | | |
| | TMA | 17 μg/m³ | 4 μg/m³ | Not detected |
| | Ammonia | 208 μg/m³ | 152 μg/m³ | 72 μg/m³ |

Referring to Table 9, when 1.0 wt % or more of the composite reducing agent was added, the sensory evaluation result was improved to grade 3.0 as a level where odor was weakly detected, and TMA was reduced by 88% or more and ammonia by 63% or more.

In addition, in order to verify the effect of the composite reducing agent from various aspects, as raw materials fed into an extrusion molding machine, 40 wt % of recycled PA6, 30 wt % of virgin PA6 (virgin polyamide 6), and 30 wt % of glass fiber were added and the composite reducing agent was added in amounts of 1.0 wt %, 1.5 wt %, and 2.0 wt % based on a total of 100 wt % thereof.

Odor sensory evaluation and quantitative analysis were performed on the manufactured recycled material specimen using the same method as above, and the results are shown in Table 10 below.

TABLE 10

| Classification | | Not added | 1.0 wt % | 1.5 wt % | 2.0 wt % |
|---|---|---|---|---|---|
| VOCs | 8 types | — | | | |
| Odor | Grade | 3.5 | 3.0 | 3.0 | 3.0 |
| | (type) | (fishy and | | | |
| | | salty odor) | | | |
| | TMA | 17 μg/m³ | 4 μg/m³ | Not detected | Not detected |
| | Ammonia | 170 μg/m³ | 127 μg/m³ | 83 μg/m³ | 42 μg/m³ |

Referring to Table 10, when 1.0 wt % or more of the composite reducing agent was added, the odor was reduced to grade 3.0, a weakly detectable level, and TMA was reduced by 76% or more and ammonia by 25% or more.

In this way, the causal substance of odor generated from the recycled PA6 was determined, the reducing material corresponding to the causal substance of odor was determined, and the optimal amount of the composite reducing agent added to the recycled PA6 was determined.

### Analysis of Composite Reducing Agent-WD-XRF

Also in Example 2 where recycled PA6 was applied, as in Example 1, pretreatment of the manufactured recycled material, quantitative analysis of the recycled material using WD-XRF, qualitative analysis of the recycled material using ED-XRF, and determination of the amount of the composite reducing agent and reproducibility using the theoretical value and quantitative analysis results of the tracer material may be performed, so a detailed process is omitted.

As is apparent from the foregoing, according to various embodiments disclosed herein, a method of applying and evaluating a reducing material corresponding to odor generated from a material to be recycled and emitted volatile organic compounds is provided.

Using the evaluation method, excessive use of the reducing material can be controlled and recycled materials having reduced odor generation and VOC emission to levels applicable to interior parts can be manufactured.

The effects of the present disclosure are not limited to the foregoing. It should be understood that the effects of the present disclosure include all effects that can be inferred from the description of the present disclosure.

As the embodiments of the present disclosure have been described above, those having ordinary skill in the art will appreciate that various modifications and alterations are possible through change, deletion or addition of components without departing from the scope and spirit of the present disclosure as described in the accompanying claims, which will also be said to be included within the scope of rights of the present disclosure.

What is claimed is:

1. A method of evaluating a composite reducing agent for a recycled material, the method comprising:
determining a reducing material to be applied to a target material;
manufacturing a composite reducing agent by mixing the reducing material and a tracer material;
determining an amount of the composite reducing agent to be added to the target material;
manufacturing a recycled material by mixing the target material and the composite reducing agent; and
analyzing the composite reducing agent in the recycled material.

2. The method of claim 1, wherein the target material comprises a waste polymer material.

3. The method of claim 1, wherein determining the reducing material comprises:
determining a causal substance of odor generated from the target material and an emitted volatile organic compounds (VOCs); and
determining the reducing material corresponding to the determined causal substance of odor and the emitted volatile organic compounds.

4. The method of claim 3, wherein determining the causal substance of odor and the emitted volatile organic compounds comprises determining the causal substance of odor and the emitted volatile organic compounds as at least one of aldehydes, amines, ketones, aromatic hydrocarbons, sulfur compounds, or fatty acids based on a chemical composition of the causal substance of odor and the emitted volatile organic compounds.

5. The method of claim 1, wherein determining the reducing material comprises selecting one type of the reducing material or determining a mixing ratio of two or more types of the reducing material.

6. The method of claim 5, wherein determining the mixing ratio of the reducing materials comprises:
mixing the two or more types of the reducing materials;
mixing the target material with the reducing materials; and
determining the mixing ratio based on results of an odor evaluation for the mixed target material and the reducing materials.

7. The method of claim 1, wherein the reducing material comprises at least one of a physical adsorbent or a chemical adsorbent.

8. The method of claim 1, wherein the reducing material comprises at least one of Zeolite 3,4A type, Zeolite X(Y) type, activated carbon, citric acid, piperazine, or peroxyacetic acid.

9. The method of claim 8, wherein:
the Zeolite 3,4A type and piperazine correspond to an aldehyde-based causal substance and volatile organic compounds,
the Zeolite X(Y) type corresponds to an amine- and ketone-based causal substance and volatile organic compounds,
the activated carbon corresponds to an aromatic hydrocarbon-based causal substance and volatile organic compounds,
the citric acid corresponds to an amine-based causal substance and volatile organic compounds, and
the peroxyacetic acid corresponds to a ketone-based causal substance and volatile organic compounds.

10. The method of claim 1, wherein the tracer material comprises metals having a molecular weight greater than carbon (C) and oxides of the metals.

11. The method of claim 1, wherein the tracer material comprises at least one of ZrO₂ or MnO₂.

12. The method of claim 1, wherein determining the amount of the composite reducing agent comprises:
mixing the target material and the composite reducing agent; and
determining the amount of the composite reducing agent based on results of an odor and volatile organic compounds evaluation for the mixed target material and the composite reducing agent.

13. The method of claim 1, wherein manufacturing the recycled material comprises adding the composite reducing agent to at least one of an extrusion process or a compounding process for the target material.

14. The method of claim 1, wherein analyzing the composite reducing agent in the recycled material comprises pretreating the recycled material.

15. The method of claim 14, wherein pretreating the recycled material comprises processing the recycled material into a specimen by mixing ash remaining after heat treatment of the recycled material with a binder.

16. The method of claim 1, wherein analyzing the composite reducing agent in the recycled material comprises determining whether the composite reducing agent is added through qualitative analysis of the recycled material.

17. The method of claim 1, wherein analyzing the composite reducing agent in the recycled material comprises determining the amount of the composite reducing agent through quantitative analysis of the recycled material.

18. The method of claim 1, wherein analyzing the composite reducing agent in the recycled material is performed using wavelength dispersive X-ray fluorescence (WD-XRF) or energy dispersive X-ray fluorescence (ED-XRF).

19. The method of claim 1, wherein analyzing the composite reducing agent in the recycled material comprises analyzing the tracer material in the recycled material and analyzing the reducing material based on analysis results of the tracer material.

20. The method of claim 1, wherein analyzing the composite reducing agent in the recycled material comprises:
performing qualitative analysis of the recycled material using energy dispersive X-ray fluorescence;
processing the recycled material into a specimen by pretreatment; and
performing quantitative analysis of the specimen using wavelength dispersive X-ray fluorescence.
